(19) United States
(12) Patent Application Publication
(10) Pub. No.: US 2025/0369048 A1
(43) Pub. Date: Dec. 4, 2025

(54) LABELING OF NUCLEIC ACID MOLECULE BY INTERSTRAND CROSSLINKED DOUBLE-STRAND DNA

(71) Applicant: HITACHI, LTD., Tokyo (JP)

(72) Inventor: Takahide Yokoi, Tokyo (JP)

(73) Assignee: HITACHI, LTD., Tokyo (JP)

(21) Appl. No.: 18/567,415

(22) PCT Filed: Jun. 15, 2022
(86) PCT No.: PCT/JP2022/023879

§ 371 Date: Dec. 6, 2023

(30) Foreign Application Priority Data
Jun. 18, 2021 (JP) 2021-101485

(51) Int. Cl. C12Q 1/6876; G01N 27/447

(52) U.S. Cl. CPC C12Q 1/6876; G01N 27/44717; G01N 27/44791; C12Q 2600/156

(57) ABSTRACT

Provided are a method and a means for improving a single base extension reaction method using capillary electrophoresis. Specifically, the present invention relates to a method for detecting presence of a target nucleic acid in a sample and/or determining a base of the target nucleic acid, the method including: preparing a sample containing or suspected of containing a target nucleic acid; preparing a primer 200 containing a double-strand nucleic acid tag 204 with an interstrand crosslink 203 and a primer nucleic acid 205 which specifically binds to the target nucleic acid; performing a single base extension reaction with the primer using the target nucleic acid as a template; and subjecting the resulting reactant to capillary electrophoresis for analysis.

### SEQUENCE LISTING

The instant application contains a Sequence Listing which has been submitted electronically in ASCII format and is hereby incorporated by reference in its entirety. Said ASCII copy, created on Feb. 4, 2025, is named 0723881637SL.txt and is 3,217 bytes in size.

### TECHNICAL FIELD

This application is a U.S. National Phase application under 35 U.S.C. § 371 of International Application No. PCT/JP2022/023879, filed on Jun. 15, 2022, which claims priority to Japanese Patent Application No. 2021-101485 filed on Jun. 18, 2021, the entire contents of which are incorporated herein by reference.

The present invention relates to a method and a means for performing genetic analysis based on the electrophoresis mobility in measurement of biomolecules, particularly nucleic acid molecules.

### BACKGROUND ART

There are various individual differences in the genome, and the difference in genome sequence between individuals is a useful biomarker as an index of disease or drug response. The method for detecting genomic variations mainly used includes detection by PCR, nucleotide sequencing using a sequencer, and a single base extension reaction analysis method (PTL 1).

Recent progress in genome science has made it possible to use a panel test of genomic variations using a massively parallel sequencer capable of performing large-scale analysis. Simultaneous analysis of a large number of genetic mutations allows for simultaneous determination of many diseases and the selection of therapies, and progress of cancer screening by liquid biopsy among them is greatly expected in the future. Liquid biopsy, that uses blood samples, is less invasive and can be used as a test target for systemic cancer. Consequently, the test has been studied as a new cancer screening (NPL 1). Meanwhile, when assuming the social implementation of cancer screening technique using the liquid biopsy, the analysis cost is a problem. The development of a low-cost technique for testing multiple mutations as an alternative to the expensive massively parallel sequencer is necessary for social implementation of cancer screening by liquid biopsy.

### CITATION LISTS

### Patent Literature

PTL 1: U.S. Pat. No. 5,888,819 B

### Non Patent Literatures

NPL 1: Cohen, J. D. et al., Science Vol. 359, pp. 926-930 (2018)
NPL 2: Coutinho, A. et al., PLOS ONE Vol. 9, No. 3, e 93292 (2014)
NPL 3: Dias-Santagata, D. et al., EMBO Molecular Medicine, Vol. 2, pp. 146-158 (2010)
NPL 4: Meagher, R. J. et al., Anal. Chem., Vol. 79, pp. 1848-1854 (2007)

### SUMMARY OF INVENTION

### Technical Problem

As an example of a technique capable of detecting various kinds of genetic mutations at low cost, there is a fragment analysis method using capillary electrophoresis (CE) (FIG. 1). This method uses a selective primer 100 which is designed to change the migration distance of electrophoresis so as to be distinguishable for each target mutation and is designed to be complementary to a gene sequence 101 containing the mutation to be detected. When there is a target genetic mutation, such a primer is used to add a dideoxynucleotide (ddNTP) modified with four kinds of fluorescent dyes 102 to 3′ terminal position of the primer corresponding to the genetic mutation (typically single nucleotide polymorphism: SNP) by polymerase synthesis reaction. Such a method is called a single base extension reaction. A primer extended for each genetic mutation with a modified molecule at the terminal by the single base extension reaction is converted into single-strand DNA, and then subjected to electrophoresis and separated using capillary electrophoresis, and the fluorescent dye at 3′ terminal is fluorescently detected. As a result, the genetic mutation is finally detected.

Since the fluorescently labeled single base extension reaction method uses the mobility of electrophoresis as an index of gene identification, it is possible to perform multiplex simultaneous detection by changing the position where the signal is detected by adjusting the length of the primer to be used or performing tag labeling for changing the mobility on the primer. In the literature of Coutinho et al. (NPL 2), simultaneous detection is carried out by the 26 plex analysis. In the literature of Dias-Santagata et al. (NPL 3), 5 to 8 kinds of mutations are detected simultaneously, and this analysis is performed 8 times, and thus a total of 58 kinds of genes are detected. However, in the methods described in NPL 2 and NPL 3, the range used for analysis is a zone of up to 120 bp in the analysis zone of the CE sequencer (FIG. 1). The CE sequencer has accuracy in separating a strand length difference of 1 base in a range of 50 to 600 bp, but the existing fluorescently labeled single base extension reaction method only uses a part of the strand length zone detected by the CE sequencer.

One of the reasons why the strand length zone detected by the CE sequencer is limited is that it is difficult to chemically synthesize >100 bp or more of oligo DNA as a primer. Another reason is that when the primer strand length increases, a long single-strand sequence portion may become a factor of non-specific binding during single base extension reaction. In order to solve these problems, mobility correction using a label other than a nucleic acid is also effective as in Meagher et al. (NPL 4), but it is not easy to synthesize many kinds of these polymers with a high degree of polymerization.

Hence, in order to extend the multiplex simultaneous detectability of the single base extension reaction method, it is required that a diverse label substance modifying the electrophoretic mobility can be synthesized, and this label substance does not cause non-specific binding to the primer. Therefore, an object of the present invention is to provide a method and a means for improving a single base extension reaction method using capillary electrophoresis.

### Solution to Problem

The present inventors have found the usefulness of interstrand crosslinked double-strand DNA molecules having various electrophoretic mobilities as primer labels, and have found that genetic analysis can be performed based on a wider difference in mobility than before without causing non-specific binding by using a selective primer to which such interstrand crosslinked double-strand DNA molecules are bound for genetic analysis, and thus they have completed the present invention.

In one aspect, the present invention provides a primer comprising: a double-strand nucleic acid tag with an interstrand crosslink; and a primer nucleic acid that specifically binds to a target nucleic acid.

In another aspect, the present invention provides a genetic analysis kit comprising the primer.

In still another aspect, the present invention provides a primer labeling kit comprising an interstrand crosslinked double-strand nucleic acid molecule, wherein the interstrand crosslinked double-strand nucleic acid molecule comprises at least one interstrand crosslinked double-strand nucleic acid unit, the interstrand crosslinked double-strand nucleic acid unit comprises: a first oligonucleotide comprising a first nucleotide sequence containing at least one interstrand crosslink-forming base and a second nucleotide sequence containing at least one interstrand crosslink-forming base; and a second oligonucleotide comprising a sequence being complementary to the second nucleotide sequence and containing a base forming an interstrand crosslink with the interstrand crosslink-forming base in the second nucleotide sequence, and a sequence being complementary to the first nucleotide sequence and containing a base forming a crosslink with the interstrand crosslink-forming base in the first nucleotide sequence, in which the first nucleotide sequence in the first oligonucleotide and the sequence complementary to the first nucleotide sequence in the second oligonucleotide form a double-strand nucleic acid.

In yet another aspect, the present invention provides a method for detecting presence of a target nucleic acid in a sample and/or determining a base of the target nucleic acid, the method comprising: preparing a sample comprising or suspected of comprising a target nucleic acid; preparing a primer comprising a double-strand nucleic acid tag with an interstrand crosslink and a primer nucleic acid which specifically binds to the target nucleic acid; performing a single base extension reaction with the primer using the target nucleic acid as a template; and subjecting the resulting reactant to capillary electrophoresis for analysis.

### Advantageous Effects of Invention

According to the present invention, as a label tag of the selective primer, a double-strand nucleic acid which can be easily synthesized with a strand length of more than 100 bp is used, whereby a detectable strand length zone in electrophoresis can be utilized in a wider range. Further, the use of, as the label tag of the selective primer, double-strand nucleic acid molecules indissociable by interstrand crosslinking can prevent non-specific binding between the primer and the label tag mixed during single base extension reaction. Therefore, according to the present invention, it is possible to detect more target nucleic acids simultaneously with high sensitivity.

### BRIEF DESCRIPTION OF DRAWINGS

FIG. 1 is a schematic view of a fragment analysis method using capillary electrophoresis.

FIG. 2 is a schematic view of a fragment analysis method using capillary electrophoresis, which uses a selective primer to which an interstrand crosslinked double-strand nucleic acid tag is bound.

FIG. 3 shows a nucleotide sequence of interstrand crosslinked double-strand DNA used in interstrand crosslink analysis.

FIG. 4 is a photograph showing an electrophoretic profile of double-strand DNA subjected to interstrand crosslink treatment.

FIG. 5 shows nucleotide sequences of an interstrand crosslinked double-strand DNA tag and a primer portion used in a fluorescent single base extension reaction test.

FIG. 6 shows graphs showing results of performing a fluorescent single base extension reaction using (A) an unlabeled primer or (B) a primer labeled with the interstrand crosslinked double-strand DNA tag, and then analyzing by capillary electrophoresis (CE).

FIG. 7 shows (A) an example of a nucleotide sequence of one unit constituting a tandem structure of the interstrand crosslinked double-strand DNA tag, and (B) a conceptual diagram of the tandem structure to be formed.

FIG. 8 is a photograph showing an electrophoretic profile of an interstrand crosslinked double-strand DNA tag having a tandem structure with a different number of units.

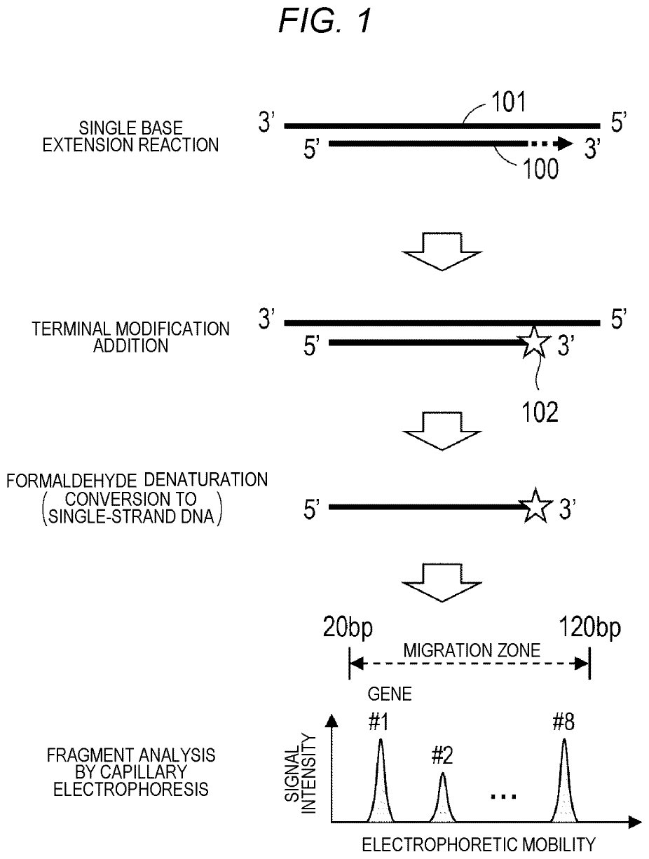

### DESCRIPTION OF EMBODIMENTS

The present invention is based on the use of an interstrand crosslinked double-strand nucleic acid tag for labeling a selective primer in a single base extension reaction of a fragment analysis method using capillary electrophoresis.

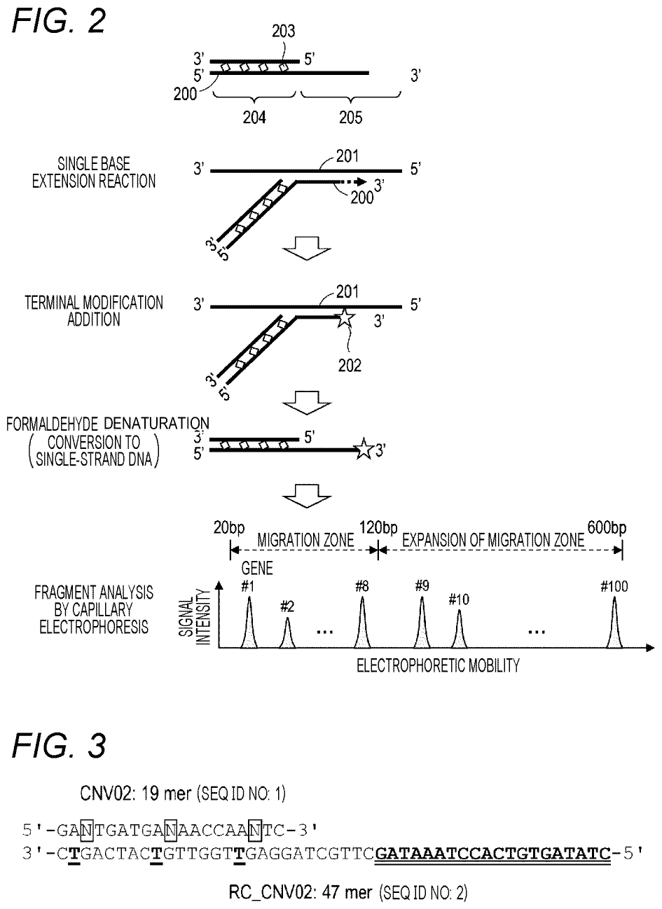

FIG. 2 shows a schematic view of a fragment analysis method using capillary electrophoresis to which the present invention is applied. A primer 200 containing a double-strand nucleic acid tag 204 having an interstrand crosslink 203 and a primer portion 205 that specifically binds to a target nucleic acid is used. A single base extension reaction may be performed using a target nucleic acid 201 as a template, and the resulting product having a modified molecule 202 at the terminal may be subjected to capillary electrophoresis (CE) to perform genetic analysis of the target nucleic acid. Changing the length of the interstrand crosslinked double-strand nucleic acid tag 204 makes it possible to label the primer distinguishably according to the difference in mobility by CE. In addition, since the tag 204 is an interstrand crosslinked double-strand nucleic acid, it is possible to prevent non-specific binding during single base extension reaction. Further, there will be no difficulty in chemical synthesis in the case of using a single-strand nucleic acid, and there will be no problems in non-target binding using a single-strand nucleic acid or in non-target binding after dissociation using a non-crosslinked double-strand nucleic acid.

Thus, in one aspect, the present invention relates to a primer containing: a double-strand nucleic acid tag with an interstrand crosslink; and a primer nucleic acid that specifically binds to a target nucleic acid.

The double-strand nucleic acid tag with an interstrand crosslink may be any of DNA, RNA, or hybrid nucleic acid as long as it is a nucleic acid having a double-strand nucleic acid structure. Preferably, the double-strand nucleic acid may be double-strand DNA.

The double-strand nucleic acid tag has at least one interstrand crosslink. In the present invention, the term “interstrand crosslink” means that one strand and the other strand in the double-strand nucleic acid are crosslinked at least at one site. The method for intramolecular crosslinking the two strands is not particularly limited as long as it is a method known in the art. Preferably, the interstrand crosslink may be performed by photocrosslinking.

For the interstrand crosslinking, for example, crosslinking molecules such as classically known nitrogen mustard, cisplatin, carmustine, mitomycin C, psoralen, trioxane (trimethyl psoralen), and malondialdehyde can be used (e.g. Guainazzi et al., Cellular and Molecular Life Sciences, 67:3683-3697, 2010). These crosslinking molecules are of a type that one crosslinking molecule enters between a base and a base, and enters between A and T or between G and C, and thus the crosslinking position is random in the entire nucleic acid molecule, and the crosslinking efficiency may be about 30 to 40%. For example, psoralen is a photocrosslinking agent that forms a photocrosslink in 5′-TA-3′ sequence by photoreaction at a photo-linking wavelength of 350 nm, and cleaves the crosslink at a photo-cleavage wavelength of 250 nm.

For the interstrand crosslinking, known crosslinking molecules that can be introduced into the oligo backbone, such as CNV-K (molecular name: 5′-O-(4,4′-Dimethoxytrityl)-1′-(3-cyanovinylcarbazol-9-yl)-2′-deoxy-β-D-ribofuranosyl-3′-[(2-cyanoethyl)-(N,N-diisopropyl)]-phosphoramidite) and CNV-D (molecular name: 3-O-(4,4′-Dimethoxytrityl)-2-N—(N-carboxy-3-cyanovinylcarbazol)-D-threonin-1-yl-O-[(2-cyanoethyl)-(N,N-diisopropyl)]-phosphoramidite) can be used (e.g. JP 4940311 B2, Yoshimura et al., ChemBioChem, 10:1473-1476, 2009; Sakamoto et al., Org. Lett., 17:936-939, 2015). These molecules can form a crosslinking point through a [2+2] cycloaddition reaction with a complementary pyrimidine base (thymine, cytosine, or uracil) at a position shifted by one base, at the starting point of irradiation with ultraviolet light (366 nm) as a reaction trigger. In addition, the crosslinking point can be introduced into the nucleic acid molecular backbone, and thus it is practically preferable that the crosslinking point can be arbitrarily designed. It is also practically important that these photocrosslinking molecules can reversibly dissociate interstrand crosslinks through excitation with ultraviolet light of a different wavelength (312 nm). The photocrosslinking molecules CNV-K and CNV-D may be particularly suitable from the viewpoint of availability, cost, and the like.

Another crosslinking molecule may be a crosslinking molecule using a Click reaction by a combination of an azido group (—N₃) with an alkyne group. Such a crosslinking point has been reported, for example, in Kocalta et al., ChemBioChem, 9:1280-1285, 2008. A known crosslinking molecule corresponding to a specific nucleotide sequence may include, for example, UTA-6026 that is specific to the sequence 5′-CAATTA-3′/3′-GTTAAT-5′ and forms a crosslink between A and G separated by 5 bases (Zhou et al., J. Am. Chem. Soc., 123:4865-4866, 2001). In addition, the known crosslinking molecule includes ImImPy (Bando et al., J. Am. Chem. Soc., 123:5158-5159, 2001) that is specific to the sequence 5′-Py(T/C)GGC(T/A)GCCPu(A/G)-3′ and forms a crosslink between bases separated by 9 bases, C8/C8′-tripyrrole-linked sequence-selective pyrrolo[2,1-c][1,4]benzodiazepine (PBD) dimer (Tiberghien et al., Bioorganic & Medicinal Chemistry Letters, 18:2073-2077, 2008) that is specific to the sequence 5′-GCTTATAATGG-3′ and forms a crosslink between bases separated by 11 bases, and the like. These sequence-specific crosslinking molecules may be advantageous in that crosslinking points can be designed.

A double-strand nucleic acid tag with an interstrand crosslink defines a migration distance (mobility) in electrophoresis. Hence, linking double-strand nucleic acid tags with different lengths to primers may make it possible to change the migration distance in electrophoresis. In capillary electrophoresis, the strand length of nucleic acid can be detected up to about 600 bp, and thus a double-strand nucleic acid tag may have a length ranging from 1 to about 590 bp, except for the strand length (10 to 30 bp) of the primer nucleic acid that specifically binds to the target nucleic acid. In addition, the double-strand nucleic acid tag that provides a distinguishable migration distance has a base length of 1 bp. For example, double-strand nucleic acid tags with different lengths of 5 bp or more, preferably 10 bp or more, may be used in combination.

In the case of a nucleic acid with an interstrand crosslink, the nucleotide sequence of the double-strand nucleic acid tag may not be particularly limited. Further, the double-strand nucleic acid tag can be chemically synthesized by a known oligonucleotide synthesis method, and may be usually synthesized using a commercially available chemical synthesizer.

The primer nucleic acid (herein also referred to as “selective primer”) that specifically binds to the target nucleic acid may be either DNA or RNA, and may be selected according to the type of the target nucleic acid and the type of polymerase used in a single base extension reaction. Preferably, the primer nucleic acid may be DNA, and the single base extension reaction may be performed using a DNA or mRNA template as the target nucleic acid.

The primer nucleic acid may be designed to have a sequence that specifically binds to the target nucleic acid (or target region), i.e. to have a sequence complementary to the target nucleic acid (or target region). Primer design methods are well known in the art, and primers that can be used in the present invention may be designed to satisfy conditions allowing for specific annealing, for example, to have a length and base composition (melting temperature) for specific annealing. For example, the length with a function as a primer may be preferably 10 bases or more, more preferably 15 to 50 bases, and still more preferably 15 to 30 bases, for example, about 20 bases. In designing the primer, it may be preferable to confirm the GC content of the primer and the melting temperature (Tm) of the primer. Tm means a temperature at which 50% of any nucleic acid strand hybridizes with its complementary strand, and it is necessary to optimize the annealing temperature in order to anneal the target nucleic acid as a template and the primer to form a double strand. Meanwhile, when the temperature is excessively lowered, a non-specific reaction occurs, and thus the temperature is desirably as high as possible. Known primer design software can be used to confirm Tm. The designed primer can be chemically synthesized by a known oligonucleotide synthesis method, and may be usually synthesized using a commercially available chemical synthesizer.

The primer according to the present invention contains an interstrand crosslinked double-strand nucleic acid tag and a selective primer, but these can be linked by any method. For example, the primer according to the present invention can be prepared by preparing a sequence in which one strand of the double-strand nucleic acid tag and a selective primer are linked directly or via a spacer, then annealing the other strand of the double-strand nucleic acid tag and forming an interstrand crosslink at least at one site of the double-strand nucleic acid portion (for example, FIG. 3). Alternatively, a double-strand nucleic acid tag with an interstrand crosslink may be prepared and then linked to a selective primer either directly or via a spacer (for example, FIG. 5). The linking method may be hydrogen bonding based on complementarity of nucleotide sequences, or may be linking using a known ligase.

To simply prepare double-strand nucleic acid tags of different lengths, for example, units of double-strand nucleic acid tags having overhangs as shown in FIG. 7, A can be linked in tandem as shown in FIG. 7, B. Changing the number of units linked in tandem may make it possible to simply prepare tags of different lengths. Further, such units of double-strand nucleic acid tags may be used, as a result of which a primer can be simply tagged and labeled only by linking (labeling) the double-strand nucleic acid tag to a selective primer. In particular, linking (labeling) double-strand nucleic acid tags of different lengths to different selective primers may make it possible to prepare a plurality of primer sets distinguishable by a difference in length (i.e. a difference in migration distance).

Therefore, in another aspect, the present invention provides a primer labeling kit containing an interstrand crosslinked double-strand nucleic acid molecule, in which the interstrand crosslinked double-strand nucleic acid molecule contains at least one interstrand crosslinked double-strand nucleic acid unit, the interstrand crosslinked double-strand nucleic acid unit contains:
a first oligonucleotide containing a first nucleotide sequence containing at least one interstrand crosslink-forming base and a second nucleotide sequence containing at least one interstrand crosslink-forming base; and
a second oligonucleotide containing a sequence being complementary to the second nucleotide sequence and containing a base forming an interstrand crosslink with the interstrand crosslink-forming base in the second nucleotide sequence, and a sequence being complementary to the first nucleotide sequence and containing a base forming a crosslink with the interstrand crosslink-forming base in the first nucleotide sequence,
in which the first nucleotide sequence in the first oligonucleotide and the sequence complementary to the first nucleotide sequence in the second oligonucleotide form a double-strand nucleic acid.

The interstrand crosslinked double-strand nucleic acid molecule contains an interstrand crosslinked double-strand nucleic acid unit, in which the interstrand crosslinked double-strand nucleic acid unit contains:
a first oligonucleotide containing a first nucleotide sequence containing at least one interstrand crosslink-forming base and a second nucleotide sequence containing at least one interstrand crosslink-forming base; and
a second oligonucleotide containing a sequence being complementary to the second nucleotide sequence and containing a base forming an interstrand crosslink with the interstrand crosslink-forming base in the second nucleotide sequence, and a sequence being complementary to the first nucleotide sequence and containing a base forming a crosslink with the interstrand crosslink-forming base in the first nucleotide sequence.

The first oligonucleotide may contain other sequences (e.g. spacer sequences) as long as it contains the first nucleotide sequence and the second nucleotide sequence. Similarly, the second oligonucleotide may contain other sequences (e.g. spacer sequences) as long as it contains the sequence complementary to the second nucleotide sequence and the sequence complementary to the first nucleotide sequence.

The interstrand crosslink-forming base may preferably be a photoresponsive interstrand crosslink-forming base as described above. For example, as a pair of an interstrand crosslink-forming base and a base forming an interstrand crosslink with the interstrand crosslink-forming base, it may be possible to use a pair of a CNV-K or CNV-D molecule and a pyrimidine base (thymine, cytosine, or uracil), the CNV-K or CNV-D molecule forming a crosslinking point through a [2+2] cycloaddition reaction with a pyrimidine base (thymine, cytosine, or uracil) at a position shifted by one base in a complementary strand. In the present specification, when the interstrand crosslink-forming base is a CNV-K or CNV-D molecule, the base forming an interstrand crosslink with the interstrand crosslink-forming base is a pyrimidine base, whereas when the interstrand crosslink-forming base is a pyrimidine base, the base forming an interstrand crosslink with the interstrand crosslink-forming base is a CNV-K or CNV-D molecule.

The interstrand crosslinked double-strand nucleic acid unit may be formed by formation of a double-strand nucleic acid between the first nucleotide sequence in the first oligonucleotide and the sequence complementary to the first nucleotide sequence in the second oligonucleotide. For example, in FIG. 7, A, when the sequence shown upper part (Core 01-Lower 01) is a first oligonucleotide and the 10 bases at 5′ side is a first nucleotide sequence, the 10 bases at 5′ side of the sequence shown lower part (Upper 01-Core 01-2) is a sequence complementary to a first nucleotide sequence in a second oligonucleotide, and the two sequences form a double-strand nucleic acid. With such a double-strand nucleic acid as a unit, the interstrand crosslinked double-strand nucleic acid molecule may contain at least one interstrand crosslinked double-strand nucleic acid unit.

In one embodiment, the interstrand crosslinked double-strand nucleic acid molecule may contain two or more of the interstrand crosslinked double-strand nucleic acid units. In this case, the two or more interstrand crosslinked double-strand nucleic acid units may be linked by formation of a double-strand nucleic acid between the second nucleotide sequence in the first oligonucleotide and the sequence complementary to the second nucleotide sequence in the second oligonucleotide. For example, in FIG. 7, B, linking the units as shown in FIG. 7, A in tandem may make it possible to prepare an interstrand crosslinked double-strand nucleic acid molecule containing a plurality of units.

In one embodiment, the primer labeling kit contains a plurality of interstrand crosslinked double-strand nucleic acid molecules including a different number of the interstrand crosslinked double-strand nucleic acid units. Thus, different selective primers can be simply and distinguishably labeled with interstrand crosslinked double-strand nucleic acid molecules of different lengths (including a different number of units).

The primer labeling kit may include, in addition to the interstrand crosslinked double-strand nucleic acid molecules, other components (buffer, ligase as necessary, etc.) used in primer labeling, instructions, and the like.

The primer according to the present invention (a primer containing a double-strand nucleic acid tag with an interstrand crosslink and a selective primer) can be used for, for example, genetic analysis, specifically, detection of a target nucleic acid, determination of a base of the target nucleic acid, and the like. The genetic analysis can be performed by any method as long as it is a method capable of identifying the target to be tested based on the difference in length. For example, capillary electrophoresis (CE) or genetic analysis by electrophoresis can be used.

Therefore, in yet still another aspect, the present invention provides a genetic analysis kit which contains: a primer containing: a double-strand nucleic acid tag with an interstrand crosslink; and a primer nucleic acid that specifically binds to a target nucleic acid. The genetic analysis kit may contain at least one primer. In a preferred embodiment, the genetic analysis kit may contain a plurality of primers containing double-strand nucleic acid tags of different lengths and primer nucleic acids that specifically bind to different target nucleic acids.

In addition to the primer, the genetic analysis kit may include a buffer constituting the reaction solution, a dNTP or ddNTP mixture (which may be labeled), enzymes (such as polymerase and reverse transcriptase), a standard sample for calibration, and the like. The primer according to the present invention may be provided as a kit, as a result of which genetic analysis can be more quickly and easily performed.

In another aspect, the present invention provides a method for detecting a target nucleic acid in a sample and/or determining a base of the target nucleic acid. The method may include: preparing a sample containing or suspected of containing a target nucleic acid; preparing a primer containing a double-strand nucleic acid tag with an interstrand crosslink and a primer nucleic acid which specifically binds to the target nucleic acid; performing a single base extension reaction with the primer using the target nucleic acid as a template; and subjecting the resulting reactant to capillary electrophoresis for analysis.

First, a sample containing or suspected of containing a target nucleic acid is prepared. The sample may not be particularly limited as long as it is a sample containing nucleic acid, and it may be possible to use any sample of a biological sample (e.g. a cell sample, a tissue sample, or a liquid sample) and a synthetic sample (e.g. a nucleic acid library such as a cDNA library). In the case of the biological sample, an organism from which the sample is derived may not be particularly limited, and it may be possible to use a sample derived from any organisms such as vertebrates (e.g. mammals, birds, reptiles, fish, and amphibians), invertebrates (e.g. insects, nematodes, and crustaceans), protists, plants, fungi, bacteria, or viruses. For example, when assuming the cancer test in humans, nucleic acid-containing samples obtained from humans to be tested, for example, whole blood, serum, plasma, saliva, urine, feces, skin tissue, and cancer tissue may be prepared.

The target nucleic acid may not be particularly limited as long as it is a nucleic acid containing a sequence to be detected or a base to be determined, and may include deoxyribonucleic acid (DNA) such as genomic DNA or cDNA, ribonucleic acid (RNA) such as messenger RNA (mRNA), and fragments thereof. In the present invention, for example, cell-free DNA (cfDNA, DNA free in blood) or circulating tumor DNA (ctDNA) may preferably be used as the target nucleic acid. The preparation of nucleic acids from a sample can be carried out by any method known in the art. For example, in the case of preparing a target nucleic acid from blood or cells, a protease such as Proteinase K, a chaotropic salt such as guanidine thiocyanate/guanidine hydrochloride, a surfactant such as Tween or SDS, or a commercially available cell lysis reagent can be used to lyse the cells, and the nucleic acids (i.e. DNA and RNA) contained in the lysate can be eluted. In the case of preparing RNA, DNA among the nucleic acids eluted by the cell lysis may be degraded by DNase to prepare a sample containing only RNA as a nucleic acid. In the case of preparing mRNA, since mRNA contains a polyA sequence, only mRNA can be captured from the RNA sample prepared as described above using a DNA probe containing a polyT sequence. To prepare such a nucleic acid, kits are available from many manufacturers, and it is possible to simply purify the target nucleic acid.

In addition, a primer containing a double-strand nucleic acid tag with an interstrand crosslink and a primer nucleic acid that specifically binds to a target nucleic acid is prepared. As described above, the double-strand nucleic acid tag may be designed to have a length distinguishable by mobility, and the primer nucleic acid may be designed to specifically bind to the target nucleic acid to cause a single base extension reaction.

In one embodiment, the present method may use a plurality of primers containing double-strand nucleic acid tags of different lengths and primer nucleic acids that specifically bind to different target nucleic acids. As described above, the base length of the double-strand nucleic acid tag that provides a distinguishable migration distance may be about 15 to 20 bases, and thus, for example, the double-strand nucleic acid tags having different lengths of 15 bases or more, preferably 20 bases or more, may be linked to different primers. In the present method, for example, 1 type to about 100 types of different target nucleic acids can be simultaneously detected.

Subsequently, a single base extension reaction with a primer may be performed using the target nucleic acid as a template. The single base extension reaction has been known in the art and is typically a single base extension reaction using a polymerase. The polymerase to be used may be selected depending on the type of template (target nucleic acid) and the type of primer to be used. For example, a DNA-dependent or RNA-dependent DNA polymerase may be used for a single base extension reaction with a DNA primer using DNA or RNA as a template, respectively.

The single base extension reaction has been widely known in the art, and for example, NPL 3 or the like describes a method of efficiently extending one base by a cycle reaction.

When the target nucleic acid is present, a selective primer that specifically binds to the target nucleic acid may hybridize, and a base may be incorporated as a substrate from 3′ terminal portion of the selective primer by a synthesis reaction by polymerase. At this time, for example, a dideoxynucleotide (ddNTP) is used as the base (substrate) to be incorporated, as a result of which the synthesis reaction may be terminated only by a single base extension. In one embodiment, a single base extension reaction may be performed using a modified base (e.g. a labeled ddNTP) as a substrate. The label may be useful for simply detecting whether or not incorporation has been conducted or for determining the type of base incorporated, and labels known in the art can be used. Such labels may include radioisotopes (such as ³²P, ¹²⁵I, and ³⁵S), fluorescent substances, and luminescent substances (such as luciferin). Fluorescent substances can be preferably used. Examples thereof may include, but not limited to, fluorescein (FITC), sulforhodamine (TR), tetramethylrhodamine (TRITC), carboxy-X-rhodamine (ROX), carboxytetramethylrhodamine (TAMRA), NED, 5-carboxyfluorescein (5-FAM), 6-carboxyfluorescein (6-FAM), 5′-hexachlorofluorescein-CE-phosphoramidite (HEX), 6-carboxy-4′,5′-dichloro-2′,7′-dimethoxyfluorescein (JOE), 5′-tetrachlorofluorescein-CE-phosphoramidite (TET), rhodamine 110 (R110), rhodamine 6G (R6G), VIC (registered trademark), ATTO system, Alexa Fluor (registered trademark) system, and Cy system. Further, examples of the fluorescent dye that does not cause a shift in the migration size may include dR110 (carboxy-dichloro rhodamine 110), dR6G (dihydro rhodamine 6G), dTAMRA (Tetramethyl rhodamine), and dROX (carboxy-X-rhodamine). For example, when trying to determine the type of base, 5 types of fluorescent substances excited and detected at different wavelengths can be used in combination to distinguish 5 types including 4 types of bases and a reference (to detect and correct the base length from the reference ladder DNA). The type of such a label, the method for introducing a label, and the like are not particularly limited, and various known methods can be used. In a preferred embodiment, a fluorescently labeled dideoxynucleotide (ddNTP) may be used as a modified base.

The presence or absence of the target nucleic acid can be determined based on whether or not the single base extension occurs, and the specific base in the target nucleic acid can be determined based on the type of base incorporated into the portion extended by one base. For example, when it is intended to detect a single nucleotide polymorphism (SNP), a selective primer that specifically binds to an upstream portion of the SNP may be designed, the selective primer may be hybridized with the target nucleic acid, and a single base extension reaction may be performed using a base with different labels as a substrate. Determining the type of the incorporated base based on the label may make it possible to detect SNP of the target nucleic acid.

After the single base extension reaction, the resulting reactant may be subjected to capillary electrophoresis (CE) for analysis. CE is a method of separating loaded components by a difference in mobility based on charge, size, shape, and the like. In the present method, since a double-strand nucleic acid tag that results in a difference in mobility is used, a target nucleic acid of interest can be detected and/or the base of the target nucleic acid can be determined from the mobility based on the type of the target nucleic acid (based on the double-strand nucleic acid tag linked to a selective primer) and the presence or absence of the target nucleic acid or the type of a specific base in the target nucleic acid (based on the single base extension reaction).

The present invention is not to be construed as being limited to the contents described in the Examples below. Those skilled in the art can easily understand that the specific configuration can be modified without departing from the spirit or gist of the present invention.

Positions, sizes, shapes, ranges, and the like of the configurations shown in the drawings are indicated for ease of understanding the invention, and in some cases do not represent the actual positions, sizes, shapes, ranges, and the like of those configurations. Thus, the present invention is not necessarily limited to the positions, sizes, shapes, ranges, and the like disclosed in the drawings.

The publications and patent publications cited herein constitute a part of the description of this specification as they are. Components expressed in the singular herein are intended to include the plural unless the context clearly dictates otherwise.

### Example 1

In this example, a selective primer containing an interstrand crosslinked double-strand DNA tag as a label was designed, and the mobility in acrylamide gel electrophoresis was examined.

The designed nucleotide sequence for interstrand crosslink analysis was shown in FIG. 3. In this example, the used base was an oligo special base for forming an interstrand crosslink through UV irradiation (CNV-D: 3-O-(4,4′-Dimethoxytrityl)-2-N—(N-carboxy-3-cyanovinylcarbazol)-D-threonin-1-yl-O-[(2-cyanoethyl)-(N,N-diisopropyl)]-phosphoramidite) disclosed in JP 4940311 B.

As shown in FIG. 3, two complementary oligo-DNA molecules, e.g. CNV02 (19 mer: SEQ ID NO: 1) and RC_CNV02 (47 mer: SEQ ID NO: 2) were designed. Each N base (each square in FIG. 3) disposed in one of the oligo-DNA molecules (SEQ ID NO: 1) is a special base forming a photocrosslink, and three photocrosslink-forming oligos were inserted in a short DNA molecule (19 mer: SEQ ID NO: 1). Each N base (CNV-D) in the CNV02 is crosslinked with each pyrimidine base (C base or T base: bold and underlined in FIG. 3) at one base upstream of RC_CNV02 (SEQ ID NO: 2) that is a complementary strand through UV irradiation at 366 nm.

For the crosslinking reaction, a 365 nm UV irradiator (ULEDN-102CT, NS Lighting Co., Ltd) was used, and the irradiation conditions were set to 62 mW and 1 second. Further, as for the solution composition during the crosslinking reaction, a KOD buffer attached to the PCR enzyme: KOD Polymerase (TOYOBO) was used at 1× concentration of the standard.

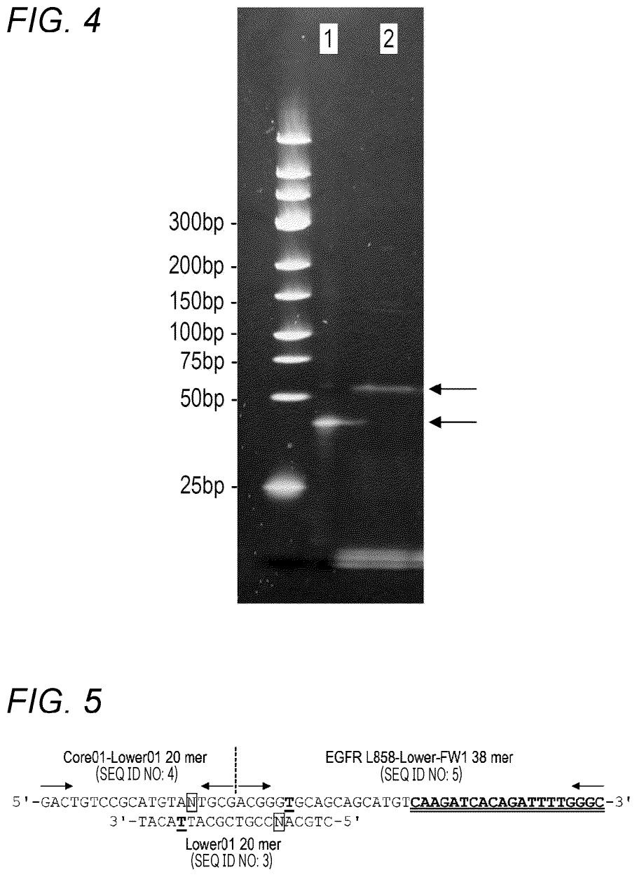

FIG. 4 is an acrylamide gel electrophoretic profile of a crosslinking reaction product. As indicated by arrows in FIG. 4, a DNA fragment image was observed at a position shorter than 50 bp in Lane 1 where RC_CNV02 (47 mer: SEQ ID NO: 2) not subjected to crosslinking was migrated, whereas a DNA fragment image was observed at a position longer than 50 bp in Lane 2 where the crosslinked product subjected to UV irradiation was migrated. This experimental result showed that interstrand crosslinks were formed by UV irradiation, the mobility of electrophoresis was changed by interstrand crosslink formation, and DNA fragments after photocrosslink formation were detected at positions of bases longer than the strand length.

### Example 2

In this example, a fluorescently labeled single base extension reaction using a primer with an interstrand crosslinked double-strand DNA tag linked as a label was verified. A target gene: EGFR gene, as a genetic mutation frequently occurring in colorectal cancer and lung cancer, was used as a target molecule to prepare a primer DNA to which a primer region specific to the genetic mutation appearing at position 788 was linked (FIG. 5).

The primer included three oligonucleotide sequences, and had a structure in which Lower01 oligo (20 mer: SEQ ID NO: 3) formed photocrosslinks with Core 01-Lower01 oligo (20 mer: SEQ ID NO: 4) on the upstream side and EGFR L858-Lower-FW1 oligo (38 mer: SEQ ID NO: 5) on the downstream side. A single-strand DNA portion of 20 bases from 3′ side of EGFR L858 Lower-FW1 oligo is a nucleotide sequence that specifically recognizes the EGFR gene to be detected (double underlined portion in FIG. 5). In this experiment, the EGFR gene was selected as an example of the target gene, but any target-specific primer was made to have a structure capable of being linked to Lower01 by using a nucleotide sequence complementary to Lower01. As shown in FIG. 5, the labeled primer may contain any single-strand DNA sequence or spacer region as long as it contains an interstrand crosslinked double-strand DNA tag portion and a selective primer DNA portion.

The fluorescent single base extension reaction using the EGFR gene sequence as a target template was performed by repeating 40 thermal cycles of (96° C., 10 seconds), (50° C., 5 seconds), and (60° C., 30 seconds) using a thermal cycler with a mixed composition: 1 μL of 10× Therminator buffer (NEB), 0.5 μL of Therminator (NEB), 1 μL of ddNTP (10 μM), 1 μL of template DNA (100 pmol/μL), 1 μL of the primer described above, and 5.5 μL of D.W. The sample solution after the reaction was subjected to a purification process with alkaline phosphatase (TAKARA), and the resulting solution was then analyzed using a CE sequencer: SeqStudio (Thermo Fisher Scientific).

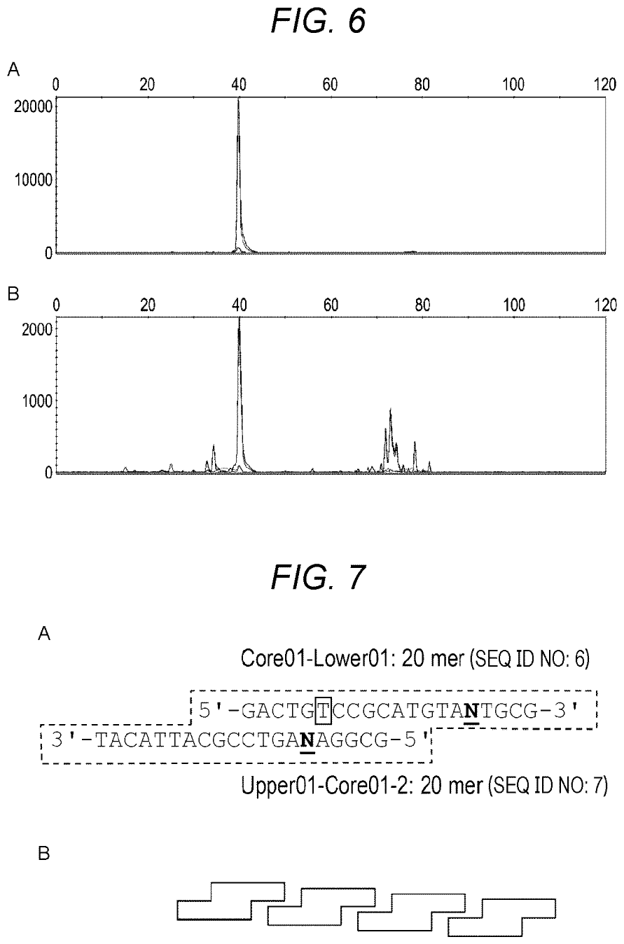

The results of fragment analysis performed by the CE sequencer were shown in FIG. 6. When an unlabeled EGFR L858-Lower-FW1 primer (SEQ ID NO: 5) was used, a fluorescent signal was detected only in the vicinity of 40 bp corresponding to a 38 mer primer length (FIG. 6, A). When using the labeled primer, a plurality of fluorescent signals was observed in the range of 70 to 80 bp in addition to the vicinity of 40 bp (FIG. 6, B). In this experiment, since the product after the crosslinking reaction is used as a primer for the fluorescently labeled single base extension reaction, the signal at the 40 bp position observed in FIG. 6, B is a signal derived from the remaining unlabeled EGFR L858-Lower-FW1. The signal in the range of 70 to 80 bp observed in FIG. 6, B is a fluorescent signal in which the migration position is changed by the label of the interstrand crosslinked double-strand DNA tag.

In addition, from the results, it was confirmed that the interstrand crosslink structure maintained the double-strand while the structure withstood 40 thermal dissociation treatments used in the fluorescently labeled single base extension reaction. Since the double-strand is maintained in the heat treatment cycle step, the interstrand crosslinked double-strand DNA of the present invention has a structure which does not bind to other primers, and it is shown that non-specific binding does not occur in the fluorescently labeled single base extension reaction.

### Example 3

In this Example, for the purpose of producing nucleic acid tags having various mobilities, the production of an interstrand crosslinked double-strand nucleic acid tag forming a tandem structure was verified. The structure of the designed double-strand nucleic acid tag (1 unit) is shown in FIG. 7, A.

In the unit of the interstrand crosslinked double-strand DNA tag shown in FIG. 7, A, the nucleotide sequence of the overhangs during double-strand formation has a sequence capable of forming a tandem structure. As shown in FIG. 7, B, it is possible to synthesize interstrand crosslinked double-strand DNA molecules having various strand lengths according to the number of linkages during UV crosslinking. This makes it possible to simply prepare a set of label tags having different lengths.

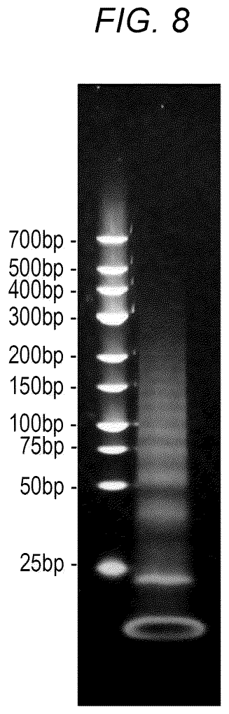

The crosslinking was performed under the same conditions as in the crosslink formation described in Example 1, and the result evaluated by acrylamide electrophoresis is shown in FIG. 8. In the electrophoretic profile, the presence of a ladder DNA fragment having about 20 bp bases as a unit was simultaneously confirmed. The present experimental results show that it is possible to synthesize double-strand nucleic acid labeled tags having various strand lengths by designing a unit structure forming a tandem structure, and it is possible to produce mobility shift tags at equal intervals by linking identical structural units.

All publications, patents, and patent applications cited herein are hereby incorporated by reference in their entirety.

### REFERENCE SIGNS LIST

100 selective primer
101, 201 template molecule
102, 202 modified molecule
200 primer
203 interstrand crosslink
204 double-strand nucleic acid tag with interstrand crosslink
205 selective primer nucleic acid

### SEQUENCE LISTING FREE TEXT

SEQ ID NOs: 1 to 7: artificial (synthetic oligonucleotide)

1. A primer comprising:
a double-strand nucleic acid tag with an interstrand crosslink; and
a primer nucleic acid that specifically binds to a target nucleic acid.

2. The primer according to claim 1, wherein the double-strand nucleic acid tag defines a migration distance in electrophoresis.

3. The primer according to claim 1, wherein the double-strand nucleic acid tag has at least one interstrand crosslink.

4. The primer according to claim 1, wherein the interstrand crosslink is formed by photocrosslinking.

5. The primer according to claim 1, wherein the double-strand nucleic acid is double-strand DNA.

6. A genetic analysis kit comprising the primer according to claim 1.

7. The kit according to claim 6, wherein the primer comprises a plurality of primers comprising double-strand nucleic acid tags of different lengths and primer nucleic acids that specifically bind to different target nucleic acids.

8. The kit according to claim 6, wherein the genetic analysis is genetic analysis by capillary electrophoresis (CE).

9. A primer labeling kit comprising an interstrand crosslinked double-strand nucleic acid molecule,
wherein the interstrand crosslinked double-strand nucleic acid molecule comprises at least one interstrand crosslinked double-strand nucleic acid unit,
the interstrand crosslinked double-strand nucleic acid unit comprises:
a first oligonucleotide comprising a first nucleotide sequence containing at least one interstrand crosslink-forming base and a second nucleotide sequence containing at least one interstrand crosslink-forming base; and
a second oligonucleotide comprising a sequence being complementary to the second nucleotide sequence and containing a base forming an interstrand crosslink with the interstrand crosslink-forming base in the second nucleotide sequence, and a sequence being complementary to the first nucleotide sequence and containing a base forming a crosslink with the interstrand crosslink-forming base in the first nucleotide sequence,
wherein the first nucleotide sequence in the first oligonucleotide and the sequence complementary to the first nucleotide sequence in the second oligonucleotide form a double-strand nucleic acid.

10. The kit according to claim 9, wherein the interstrand crosslink-forming base is a photoresponsive interstrand crosslink-forming base.

11. The kit according to claim 9, wherein the interstrand crosslinked double-strand nucleic acid molecule comprises two or more of the interstrand crosslinked double-strand nucleic acid units, and the two or more interstrand crosslinked double-strand nucleic acid units are linked by formation of a double-strand nucleic acid between the second nucleotide sequence in the first oligonucleotide and the sequence complementary to the second nucleotide sequence in the second oligonucleotide.

12. The kit according to claim 9, comprising a plurality of interstrand crosslinked double-strand nucleic acid molecules comprising a different number of the interstrand crosslinked double-strand nucleic acid units.

13. A method for detecting presence of a target nucleic acid in a sample and/or determining a base of the target nucleic acid, the method comprising:
preparing a sample comprising or suspected of comprising a target nucleic acid;
preparing a primer comprising a double-strand nucleic acid tag with an interstrand crosslink and a primer nucleic acid which specifically binds to the target nucleic acid;
performing a single base extension reaction with the primer using the target nucleic acid as a template; and
subjecting the resulting reactant to capillary electrophoresis for analysis.

14. The method according to claim 13, wherein the primer comprises a plurality of primers comprising double-strand nucleic acid tags of different lengths and primer nucleic acids that specifically bind to different target nucleic acids.

15. The method according to claim 13, wherein the single base extension reaction is performed using a modified base as a substrate.

16. The method according to claim 15, wherein the modified base comprises a fluorescently labeled dideoxynucleotide (ddNTP).


SEQUENCE LISTING (7 sequences)

SEQ ID NO: 1 - DNA, 19 bp, Artificial
  synthetic oligonucleotide
  misc_feature (3)..(3): n = 3-O-(4,4′-Dimethoxytrityl)-2-N-(N-carboxy-3-cyanovinylcarbazol)-D -threonin-1-yl-O-[(2-cyanoethyl)-(N,N-diisopropyl)]-phosphoramidi te
  misc_feature (10)..(10): n = 3-O-(4,4′-Dimethoxytrityl)-2-N-(N-carboxy-3-cyanovinylcarbazol)-D -threonin-1-yl-O-[(2-cyanoethyl)-(N,N-diisopropyl)]-phosphoramidi te
  misc_feature (17)..(17): n = 3-O-(4,4′-Dimethoxytrityl)-2-N-(N-carboxy-3-cyanovinylcarbazol)-D -threonin-1-yl-O-[(2-cyanoethyl)-(N,N-diisopropyl)]-phosphoramidi te
gantgatganaaccaantc

SEQ ID NO: 2 - DNA, 47 bp, Artificial
  synthetic oligonucleotide
ctatagtgtcacctaaatagcttgctaggagttggttgtcatcagtc

SEQ ID NO: 3 - DNA, 20 bp, Artificial
  synthetic oligonucleotide
  misc_feature (6)..(6): n = 3-O-(4,4′-Dimethoxytrityl)-2-N-(N-carboxy-3-cyanovinylcarbazol)-D -threonin-1-yl-O-[(2-cyanoethyl)-(N,N-diisopropyl)]-phosphoramidi te
ctgcanccgtcgcattacat

SEQ ID NO: 4 - DNA, 20 bp, Artificial
  synthetic oligonucleotide
  misc_feature (16)..(16): n = 3-O-(4,4′-Dimethoxytrityl)-2-N-(N-carboxy-3-cyanovinylcarbazol)-D -threonin-1-yl-O-[(2-cyanoethyl)-(N,N-diisopropyl)]-phosphoramidi te
gactgtccgcatgtantgcg

SEQ ID NO: 5 - DNA, 38 bp, Artificial
  synthetic oligonucleotide
acgggtgcagcagcatgtcaagatcacagattttgggc

SEQ ID NO: 6 - DNA, 20 bp, Artificial
  synthetic oligonucleotide
  misc_feature (16)..(16): n = 3-O-(4,4′-Dimethoxytrityl)-2-N-(N-carboxy-3-cyanovinylcarbazol)-D -threonin-1-yl-O-[(2-cyanoethyl)-(N,N-diisopropyl)]-phosphoramidi te
gactgtccgcatgtantgcg

SEQ ID NO: 7 - DNA, 20 bp, Artificial
  synthetic oligonucleotide
  misc_feature (6)..(6): n = 3-O-(4,4′-Dimethoxytrityl)-2-N-(N-carboxy-3-cyanovinylcarbazol)-D -threonin-1-yl-O-[(2-cyanoethyl)-(N,N-diisopropyl)]-phosphoramidi te
gcgganagtccgcattacat